(19) United States
(12) Plant Patent Application Publication
(10) Pub. No.: US 2010/0031408 P1
(43) Pub. Date: Feb. 4, 2010

(54) Dianthus plant named 'WP08 UNI02'

(50) ;

(76) Inventor: Carolyn Grace Bourne, Dawlish (GB)

Correspondence Address: PLANT HAVEN, INC., P.O. BOX 3056, SANTA BARBARA, CA, 93130, US

(21) Appl. No.: 12/460,777

(22) Filed: Jul. 24, 2009

(30) Foreign Application Priority Data
Jul. 30, 2008 (EP) 2008/1676

(51) Int. Cl. A01H 5/00

(52) U.S. Cl. PLT/279

(57) ABSTRACT

A new cultivar of Dianthus plant named ‘WP08 UNI02’ whose flowers are pure white with a crimson eye with markedly fimbriated petals. In combination these traits set ‘WP08 UNI02’ apart from all other existing varieties of Dianthus known to the inventor.

### GENUS

Dianthus

### SPECIES

×hybrida

### DENOMINATION

‘WP08 UNI02’

### BACKGROUND OF THE INVENTION

This application claims the benefit of priority under 35 U.S.C. 119(f) of the application for a grant of European Community Plant Breeders Rights which was filed for the instant plant variety on Jul. 30, 2008, Application Number 2008/1676.

The present invention relates to a new and distinct cultivar of hardy garden pink or carnation that is grown for use as a flowering plant for pots and containers and for planting in the garden and landscape. The new cultivar is known botanically as Dianthus×hybrida and will be referred to hereinafter by the cultivar name ‘WP08 UNI02’.

‘WP08 UNI02’ is a complex hybrid plant that is the product of a breeding program started by the inventor in 1985. The breeding program is conducted in a cultivated area of Houndspool, Dawlish, Devon, United Kingdom. The inventor selected ‘WP08 UNI02’ in 2004 for its early season flowering and for the color combination of the flowers, which are pure white with a crimson eye. The flowers of ‘WP08 UNI02’ are deeply fimbriated.

‘WP08 UNI02’ resulted from the open pollination between Dianthus ‘Devon Winnie’ (U.S. Plant Pat. No. 14,893) as female parent plant and an unknown, unreleased seedling as male parent plant.

‘WP08 UNI02’ is distinguishable from the female parent as follows: The height of the female parent is approximately two-thirds of the height of ‘WP08 UNI02’. The flowers of the female parent are smaller in height. The flowers of the female parent are dark pink in color and are borne on shorter stems than the flowers of ‘WP08 UNI02’.

‘WP08 UNI02’ cannot be described in comparison with the unknown male parent.

The variety of Dianthus which is considered by the inventor to most closely resemble ‘WP08 UNI02’ is Dianthus ‘WP05 YVES’ (U.S. Plant Pat. No. 18,828). ‘WP08 UNI02’ is distinguishable from this variety as follows: Mature plants of ‘WP05 YVES’ are approximately 5 cm shorter in height than plants of ‘WP08 UNI02’. The flowers of ‘WP08 UNI02’ are larger and more fimbriated compare to the flowers of ‘WP05 YVES’.

Asexual reproduction of ‘WP08 UNI02’ was first accomplished by the inventor in 2004 in a cultivated area of Houndspool, United Kingdom. The method of asexual propagation used was vegetative cuttings. Since that time the characteristics of the new cultivar have been determined stable and are reproduced true to type in successive generations.

### SUMMARY OF THE INVENTION

The following traits have been repeatedly observed and represent the characteristics of the new Dianthus cultivar ‘WP08 UNI02’. These traits in combination distinguish this cultivar from all other commercial varieties known to the inventor. ‘WP08 UNI02’ has not been tested under all possible conditions and phenotypic differences may be observed with variations in environmental, climatic and cultural conditions, however, without any variance in genotype.
1. Dianthus ‘WP08 UNI02’ exhibits a compact habit.
2. Dianthus ‘WP08 UNI02’ exhibits flowers that are pure white with a crimson eye.
3. The flowers of Dianthus ‘WP08 UNI02’ are markedly fimbriated.
4. Dianthus ‘WP08 UNI02’ blooms from April through October.
5. Dianthus ‘WP08 UNI02’ exhibits grey-green foliage.
6. At nine months of age, the height of the foliage mound of Dianthus ‘WP08 UNI02’ is 20 cm to 30 cm, and the width is 15 cm to 20 cm.
7. Dianthus ‘WP08 UNI02’ is perennial and is hardy to −15° C.
8. Dianthus ‘WP08 UNI02’ is suitable for use as a flowering plant in pots and containers.

### BRIEF DESCRIPTION OF THE DRAWINGS

The accompanying color drawings illustrate the overall appearance of the new Dianthus variety ‘WP08 UNI02’ showing colors as true as it is reasonably possible to obtain in colored reproductions of this type. Colors in the drawings may differ from the color values cited in the detailed botanical description, which accurately describe the actual colors of the new variety ‘WP08 UNI02’.

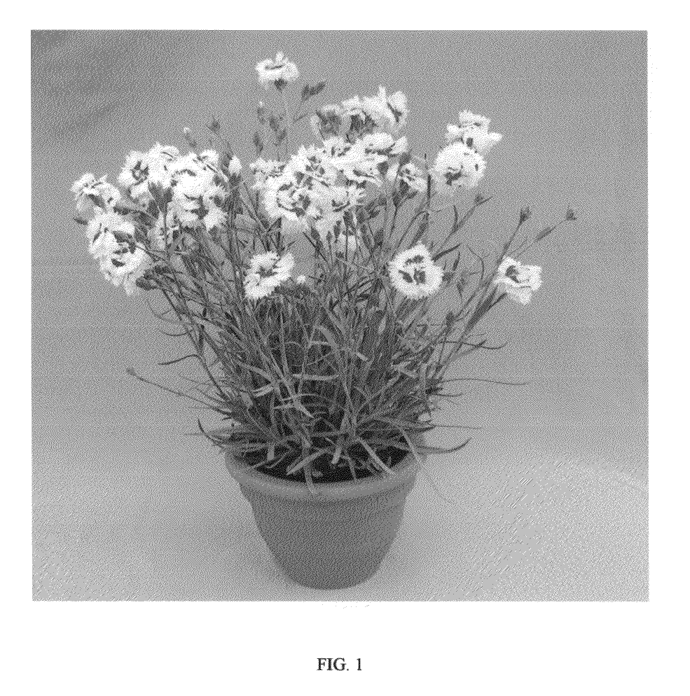

The drawing labeled as FIG. 1 depicts an entire flowering plant.

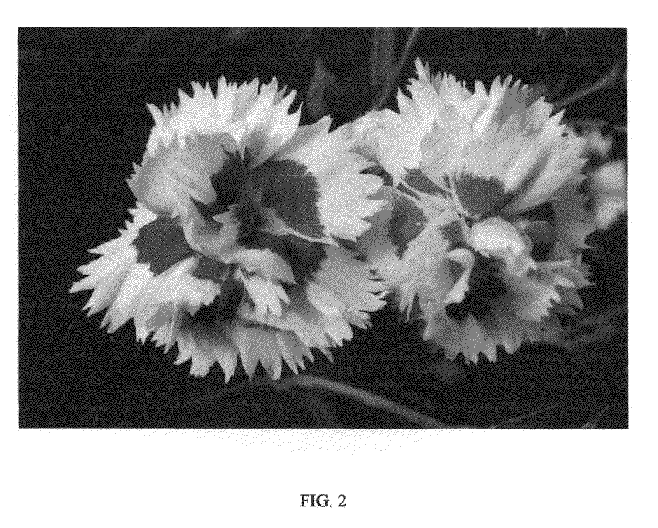

The drawing labeled as FIG. 2 depicts a close-up view of the flower.

All drawings were made of 9 months old plants which have been grown in 1.5-litre containers in a cold glasshouse in Devon, United Kingdom. No chemicals were used to treat the plants. All drawings were made using conventional techniques and although colors may appear different from actual colors due to light reflectance they are as accurate as possible by conventional photography.

### DETAILED BOTANICAL DESCRIPTION OF THE PLANT

The following is a detailed description of the new cultivar ‘WP08 UNI02’. Data was collected in spring from 1.5-litre container plants in spring, and grown indoors in Devon, United Kingdom. The color determinations are in accordance with the 2002 edition of The Royal Horticultural Society Colour Chart of The Royal Horticultural Society, London, England, except where general color terms of ordinary dictionary significance are used. The colors of stems, leaves, calyx, and epicalyx have been noted on plants which retain the characteristic waxy bloom of Dianthus. No chemicals were used to treat the plants. Growing conditions are typical to other Dianthus.
Botanical classification:
Family: Caryophyllaceae
Genus: Dianthus
Species: ×hybrida
Denomination: ‘WP08 UNI02’
Parentage: A complex Dianthus hybrid whose immediate parents are unreleased hybrids from the inventor's breeding program, identified as follows:
Male parent: Unknown
Female parent: Dianthus ‘Devon Winnie’ (U.S. Plant Pat. No. 14,893)
Plant type: Flowering perennial.
Use of plant: As a flowering plant in containers, window boxes, planters, pots and the garden.
Bloom period: April-October in Devon, UK
Plant habit: A compact mound with many branches and stems upright
Height of plant: 20 to 30 cm after 9 months
Width of plant: 15 to 20 cm after 9 months
Hardiness: Has survived in temperatures of minus 15 Centigrade, equivalent to USDA Hardiness Zone 7.
Root system: many fibrous roots
Propagation method: vegetative stem cuttings
Cultural requirements: Grow in full sun, in well drained and moderately fertile soil, keep well fertilized and watered.
Diseases and pests: No particular resistance or susceptibility to Dianthus diseases and pests are known to the inventor
Time to root cuttings: 5 weeks is required to produce roots on a vegetative cutting.
Temperature for rooting: A minimum of 15° C. air temperature and 21° C. base temperature at root level is required to produce roots on the cuttings.
Facilities for rooting: Mist propagation unit is required for satisfactory rooting of cuttings.
Crop time: 3 months starting in the Spring and 6 months starting in the Fall.
Plant stem:
   Shape.—Cylindrical.
   Height.—10 to 15 cm.
   Diameter.—5 mm.
   Surface.—Glabrous and glaucous.
   Color.—145C.
   Branching.—Numerous shoots from the axils of the lower leaves.
   Internode length (between nodes).—3 to 5 mm.
Leaf:
   Type.—Evergreen.
   Shape.—Linear, straight.
   Division.—Simple.
   Apex.—Acute.
   Base.—Decurrent.
   Venation.—Not prominent.
   Margins.—Entire.
   Arrangement.—Opposite and spiraling on stem.
   Attachment.—Sheathing.
   Surface adaxial.—Glaucous.
   Surface abaxial.—Glaucous.
   Fragrance.—Absent.
   Dimensions.—Length: 95 to 115 mm Width: 4 to 5 mm.
   Color.—Adaxial surface: N138B-C Abaxial surface: N138B-C.
Inflorescence:
   Flower stem.—Length: 20 to 25 cm Width: 3 mm Stem strength: Erect Stem color: 122B to 133C.
   Inflorescence.—Cymose.
   Type.—Salviform, symmetrical.
   Development.—Singly, in sequence from the uppermost bud.
Buds:
   Color.—138A, apex 137A.
   Dimensions.—11 cm in length (height) and 10 cm in width.
   Shape.—Conical.
Flowers:
   Number.—5 to 7 per stem.
   Dimensions.—Length (including calyx): 34 to 36 mm Diameter: 42 to 47 mm.
   Flower shape.—Circular, asymmetric.
   Flower profile.—Concave.
   Lastingness.—10 days at 20° C., on or off the plant.
   Fragrance.—Medium perfume.
   Flowering period.—April to October, in Devon, UK.
Corolla:
   Shape (upper part of corolla).—Convex.
   Shape (lower part of corolla).—Concave.
   Petal arrangement.—Persistent, semi-double, overlapping.
   Petal margin.—Serrate.
   Indentation.—Up to 5 mm deep.
   Petal texture.—Soft.
   Flower type.—Double.
   Petal number.—15 petals and from 5 to 10 petaloid stamens.
   Petal dimension.—Length: 37 mm Width: 26 to 29 mm.
   Color pattern.—Band.
   Color of upper surface.—Ground color of blade: White Color of band across blade: 60A Color of middle of strap: 145C and 145B Color of base of strap: 145C and 145D.
   Color of lower surface.—Ground color of blade: White Color of band across blade: White Color of middle of strap: 145C and 145B Color of base of strap: 145C and 145D.
   Petal shape.—Obdeltoid.
   Petal surface.—Glabrous, flat.
Calyx:
   Shape.—Cylindrical.
   Longitudinal profile.—Convex.
   Dimensions.—Length: 22 mm Diameter: 9 to 10 mm.
   Sepals.—Fused.
   Sepals number.—5.
   Sepals color.—143A on the top and 143B on the base.
   Anthocyanin.—On edge of the lobe and sometime as flush on the calyx.
   Splitting.—Not observed.
Epicalyx:
   Bracts number.—1 pair of two.
   Shape of outer lobe.—Acuminate.
   Dimensions.—Width: 7 to 8 mm Length: 9 to 10 mm.
   Color.—139B on the tip and 139C on the center.
   Anthocyanin.—Absent.
Bracteoles:
   Number.—1 pair of two.
   Dimensions.—Width: 8 to 10 mm Length: 9 to 10 mm.
   Color.—139B on the tip and 139C on the center.
   Anthocyanin.—Absent.
Reproductive organs:
   Stamens.—Number: Up to 5 (+ up to 5 stamens petaloids) Color of filaments: White Dimensions: Length: 15 to 18 mm Diameter: 0.5 mm.
   Anthers.—Number: Up to 5. Color: 73B to 73C Dimensions: Length: 2 to 3 mm Diameter: 1 mm Attachment: Looks dorsifixed (but not sure as the anthers are deformed).
   Pollen.—Present occasionally.
   Pollen color.—157A.
   Style.—Number: 2 Shoulder: Absent Color: White Length: 25 to 30 mm Width: 1 mm.
   Stigma.—Number: 2 (1 per style) Color: White Dimensions: 5 to 10 mm above the flower at maturity.
   Ovary.—Position: Superior Dimensions: Length: 7 mm Diameter: 4 mm Shape: Spindle Color (upper surface): 145B Color (lower surface): 145D.
   Seed.—Seed has not been observed.

1. A new and distinct cultivar of Dianthus plant named ‘WP08 UNI02’ as described and illustrated herein.
